SEQUENCE LISTING (9 sequences)

SEQ ID NO: 1 - DNA, 6718 bp, synthetic construct
  source 1..6718: mol_type = other DNA
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccgggtcgtgcaacttcagactcttacggaggcgatggaccaaaaacaactacaatcaagcagatcaccttgtacaccaccatagaaaaggcccaccctcagccatggctatcagtgttgttgatctatttagcatcggtatcggaccatcatcctcacataccgtcggccccatgagagccgccctcacgtatatctctgaatttcccagctcgcatgtcgatatcacgttgcacggatcccttgccgccaccggtaaaggccactgcactgaccgggcggtattactgggtctggtgggatgggaaccaacgatagttcccattgatgctgcaccctcacccggcgcgccgattcctgcgaaaggttctgtgaacgggccaaagggaacggtgtcgtattccctgacgtttgatcctcatcctcttccagaacaccccaatgccgttacctttaaaggatcaaccacaaggacttatttgtcggtgggtggtgggttcattatgacgtccgactaatggtgctgcggggattattccggcggtgatgcactatgcgcgggattttttgacaggttttggggcggagcaggcgcggacgtttttgtataccgcgggtgcggtgggcatcatcattaaggaaaatgcctcgatctctggcgcggaggtggggtgtcagggtgaggttggttcagcgtccgcgatggcggctgccgggttgtgtgcagtcttaggtggttctccgcaacaggtggaaaacgccgcggagattgcgttggagcacaatttgggattgacgtgcgatccggtgggcgggttagtgcagattccgtgtattgaacgcaacgctattgctgccatgaagtccatcaatgcggcaaggcttgcccggattggtgatggcaacaatcgcgtgagtttggatgatgtggtggtcacgatggctgccaccggccgggacatgctgaccaaatataaggaaacgtcccttggtggtttggcaaccaccttgggcttcccgcctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgg

SEQ ID NO: 2 - DNA, 38 bp, synthetic construct
  source 1..38: mol_type = other DNA
attcgagctcggtacccgggtcgtgcaacttcagactc

SEQ ID NO: 3 - DNA, 37 bp, synthetic construct
  source 1..37: mol_type = other DNA
cgcagcaccattagtcggacgtcataatgaacccacc

SEQ ID NO: 4 - DNA, 37 bp, synthetic construct
  source 1..37: mol_type = other DNA
ggtgggttcattatgacgtccgactaatggtgctgcg

SEQ ID NO: 5 - DNA, 38 bp, synthetic construct
  source 1..38: mol_type = other DNA
caagcttgcatgcctgcaggcgggaagcccaaggtggt

SEQ ID NO: 6 - DNA, 1146 bp, Moorea producens
  source 1..1146: mol_type = genomic DNA
atgtcggaaaaaattgttaattcctggaatgaatgggatgaattggaagaaatggtggtaggaattgcagactatgctagctttgaaccaaaagaaccagggaatcatccgaaattaagaaatcaaaatttagcggaaatcattcctttccccagtggacctaaagaccctaaagtccttgaaaaagctaatgaagaattaaatggactggcttatttattaaaagaccacgatgtgatagtaagaagacccgaaaaaattgattttactaaatctctaaaaacaccttactttgaagttgcaaatcaatactgtggagtctgtcctcgggatgtcatgattacctttgggaatgaaatcatggaagcgactatgtcgaagagagctagattttttgaatacttaccttaccggaaattggtctatgaatattggaataaagacgagcatatgatttggaatgctgcgcctaaaccgactatgcaggatagtatgtatctagagaatttctgggagctgtctttagaagaacgatttaagcgtatgcatgattttgaattttgtattacacaagatgaagtaatttttgatgcggctgactgtagcagattaggaaaggatatattagttcaggaatcgatgacaacaaatagaacaggaattcggtggttaaaaaagcacctagaaccaagaggatttcgggttcaccctgttcattttccccttgattttttcccctcacacattgactgtacgtttgttcctttgcgaccaggtcttattttgacaaaccctgaaagacctatacgggaagaggaggagaagatttttaaagagaatggctgggagttgatcacagttcctcaaccgacttgctcgaatgatgaaatgccaatgttttgccagtccagtaagtggttgtcaatgaatgttctgagtatatcaccgacaaaggttatctgtgaggaaagagaaaaacctctccaagaattgttggataagcatggatttgaggtttttcctttaccctttagacatgtctttgaatttggggggtcttttcattgtgcaacttgggatattcgccgaaaaggtgagtgtgaagattatttaccaaatttaaactatcaaccgatttgtggttaa

SEQ ID NO: 7 - protein, 381 aa, Moorea producens
MSEKIVNSWNEWDELEEMVVGIADYASFEPKEPGNHPKLRNQNLAEIIPFPSGPKDPKVLEKANEELNGLAYLLKDHDVIVRRPEKIDFTKSLKTPYFEVANQYCGVCPRDVMITFGNEIMEATMSKRARFFEYLPYRKLVYEYWNKDEHMIWNAAPKPTMQDSMYLENFWELSLEERFKRMHDFEFCITQDEVIFDAADCSRLGKDILVQESMTTNRTGIRWLKKHLEPRGFRVHPVHFPLDFFPSHIDCTFVPLRPGLILTNPERPIREEEEKIFKENGWELITVPQPTCSNDEMPMFCQSSKWLSMNVLSISPTKVICEEREKPLQELLDKHGFEVFPLPFRHVFEFGGSFHCATWDIRRKGECEDYLPNLNYQPICG

SEQ ID NO: 8 - DNA, 7061 bp, synthetic construct
  source 1..7061: mol_type = other DNA
gacagtaagacgggtaagcctgttgatgataccgctgccttactgggtgcattagccagtctgaatgacctgtcacgggataatccgaagtggtcagactggaaaatcagagggcaggaactgcagaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcctgcagggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatgggcctgcaggcctcagcgcgattcaggcctggtatgagtcagcaacaccttcttcacgaggcagatctcagcgcccccccccccctagcttgtctacgtctgatgctttgaatcggacggacttgccgatcttgtatgcggtgatttttccctcgtttgcccactttttaatggtggccggggtgagagctacgcgggcggcgacctgctgcgctgtgatccaatattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagttcctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctagattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttgacaatggctgatttcatcaggaatggaactgtcatgctgttatgtgcctggctcctaatcaaagctggggacaatgggttgccccgttgatctgatctagttcggattggcggggcttcactgtatctgggggtggcatcgtgaatagattgcacaccgtagtgggcagtgtgcacaccatagtggccatgagcaccaccacccccagggacgccgacggcgcgaagctctgcgcctggtgcggctcggagatcaagcaatccggcgtcggccggagccgggactactgccgccgctcctgccgccagcgggcgtacgaggcccggcgccagcgcgaggcgatcgtgtccgccgtggcgtcggcagtcgctcgccgagatacgtcacgtgacgaaatgcagcagccttccattccgtcacgtgacgaaactcgggccgcaggtcagagcacggttccgcccgctccggccctgccggacccccggcatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatccgagtccaatgtgtccgtgttcacctcagcggccgcaccggtcggggtaccttgacaaatactttccaatggggtacaatggcgcagtgccatccactccgaggttgataccagcctcctcggtggaatgatcatccgcgtcggagacgaagtaattgacggcagcacctcggggagacctcaaatggtctcgcgttaacttaaggtaccgaattctaagcttcacaaaaaaaaaccccgcccctgacagggcggggttttttttactcagcatgtggatggccagtggtcggaccgagcgctttaattaattagcatggtgacacaagcacagtat

SEQ ID NO: 9 - DNA, 8312 bp, synthetic construct
  source 1..8312: mol_type = other DNA
gacagtaagacgggtaagcctgttgatgataccgctgccttactgggtgcattagccagtctgaatgacctgtcacgggataatccgaagtggtcagactggaaaatcagagggcaggaactgcagaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcctgcagggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatgggcctgcaggcctcagcgcgattcaggcctggtatgagtcagcaacaccttcttcacgaggcagatctcagcgcccccccccccctagcttgtctacgtctgatgctttgaatcggacggacttgccgatcttgtatgcggtgatttttccctcgtttgcccactttttaatggtggccggggtgagagctacgcgggcggcgacctgctgcgctgtgatccaatattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagttcctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctagattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttgacaatggctgatttcatcaggaatggaactgtcatgctgttatgtgcctggctcctaatcaaagctggggacaatgggttgccccgttgatctgatctagttcggattggcggggcttcactgtatctgggggtggcatcgtgaatagattgcacaccgtagtgggcagtgtgcacaccatagtggccatgagcaccaccacccccagggacgccgacggcgcgaagctctgcgcctggtgcggctcggagatcaagcaatccggcgtcggccggagccgggactactgccgccgctcctgccgccagcgggcgtacgaggcccggcgccagcgcgaggcgatcgtgtccgccgtggcgtcggcagtcgctcgccgagatacgtcacgtgacgaaatgcagcagccttccattccgtcacgtgacgaaactcgggccgcaggtcagagcacggttccgcccgctccggccctgccggacccccggcatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatccgagtccaatgtgtccgtgttcacctcagcggccgcaccggtcggggtaccttgacaaatactttccaatggggtacaatggcgcagtgccatccactccgaggttgataccagcctcctcggtggaatgatcatccgcgtcggagacgaagtaattgacggcagcacctcgggcaaactcgagcgtctgcgggcaagcttcgcataaagacacgacgaattagacaacattagtaatgctggaagaaacaaccgagagcaggaagaacatgtcagaaaagattgttaactcctggaacgaatgggacgaacttgaagaaatggtggttggcatcgcggactacgcttccttcgagccaaaagaaccaggtaaccaccctaagctacgcaaccagaacctggccgagatcatcccattcccatccggcccaaaggacccaaaggttctcgaaaaagcaaacgaagagctgaacggcctcgcatacctcctaaaggaccacgatgttatcgtccgccgcccagaaaaaattgatttcactaagtcccttaaaaccccatacttcgaggtcgctaaccagtactgcggcgtttgcccacgcgacgttatgattaccttcggtaatgagattatggaggctaccatgagcaagcgtgcacgcttcttcgagtacttgccataccgtaagctcgtctacgagtactggaacaaggatgagcatatgatctggaacgctgctccaaagcctacgatgcaggactccatgtacttggagaacttctgggagctgtccctcgaggagcgcttcaagcgcatgcacgacttcgaattttgcattacccaggacgaggttatcttcgacgcagctgactgctcccgcctgggcaaggacatcctggtccaggagtccatgaccaccaaccgcaccggcattcgttggttgaagaagcacctcgaacctcgcggtttccgcgttcacccagtccacttccctctcgacttcttcccttctcacattgactgcactttcgtccctctccgtcccggcctcatccttaccaacccagagcgcccaatccgcgaagaagaagaaaagatcttcaaggagaacggctgggagcttatcaccgtcccacaacctacctgctccaacgacgaaatgcctatgttctgccagtcctcgaagtggctcagcatgaacgtcctttctatctccccaacaaaggttatttgcgaagagcgtgaaaagccactacaggaactcctggacaagcacggcttcgaagtcttcccactcccattccgccacgttttcgaattcggcggctcctttcactgcgccacttgggacatccgccgcaagggcgaatgcgaagactacctcccaaacctgaactaccagcctatctgcggctaataagctcggtacccggggatccgacgtcgcgttaacttaaggtaccgaattctaagcttcacaaaaaaaaaccccgcccctgacagggcggggttttttttactcagcatgtggatggccagtggtcggaccgagcgctttaattaattagcatggtgacacaagcacagtat

(19) United States
(12) Patent Application Publication
(10) Pub. No.: US 2026/0218253 A1
(43) Pub. Date: Jul. 30, 2026

(54) FERMENTATIVE PRODUCTION GUANIDINOACETIC ACID (GAA) FROM SERINE BY ATTENUATING L SERINE AMMONIA LYASE ACTIVITY IN MICROORGANISMS

(71) Applicant: Evonik Operations GmbH, Essen (DE)

(72) Inventors: Steffen Schaffer, Herten (DE); Martina Carrillo Camacho, Hanau (DE); Frank Schneider, Halle (DE); Mirja Wessel, Bochum (DE)

(73) Assignee: Evonik Operations GmbH, Essen (DE)

(21) Appl. No.: 19/146,607

(22) PCT Filed: Dec. 29, 2023
(86) PCT No.: PCT/EP2023/087969

§ 371 Date: Jul. 9, 2025

(30) Foreign Application Priority Data
Jan. 9, 2023 (EP) 23150745.0

(51) Int. Cl. C12P 13/04; C12N 9/00; C12N 9/10; C12N 9/88; C12N 15/52

(52) U.S. Cl. CPC C12P 13/04; C12N 9/1014; C12N 9/1018; C12N 9/88; C12N 9/93; C12N 15/52; C12Y 201/01002; C12Y 201/02001; C12Y 201/03003; C12Y 201/04001; C12Y 403/01017; C12Y 403/02001; C12Y 603/04005; C12Y 603/04016

(57) ABSTRACT

A microorganism has been modified to express genes encoding proteins having arginine:glycine amidinotransferase (AGAT) activity as well as modified to increase the production of glycine from L-serine and a method produces guanidinoacetic acid (GAA) through the fermentation of such microorganism and a method produces creatine.

The present invention provides a microorganism which has been modified to express genes encoding proteins having arginine:glycine amidinotransferase (AGAT) activity and to increase the production of glycine from L-serine and a method of producing guanidinoacetic acid (GAA) through the fermentation of such microorganism as well as a method for producing creatine.

Guanidinoacetic acid (GAA) is the direct precursor of creatine, which is active in vertebrate energy homeostasis. GAA is used in feed formulations to improve feed conversion rates, animal health and meat quality. GAA is formed in vertebrates and occasionally also in bacterial metabolism (secondary metabolism) from L-arginine and glycine. This step is catalyzed by the enzyme arginine:glycine amidinotransferase (AGAT) [E.C. 2.1.4.1], whereby L ornithine is produced as a by-product.

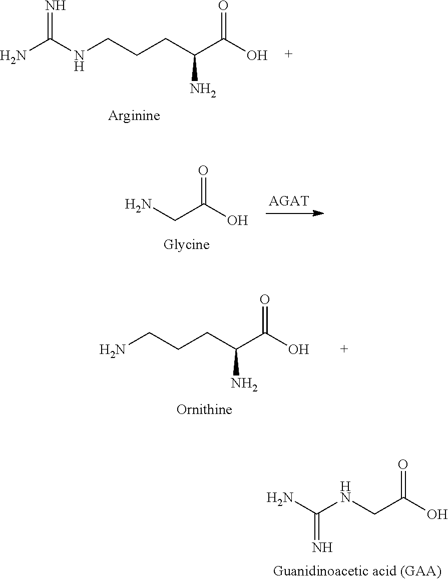 [SMILES: N=C(N)NCCC[C@H](N)C(=O)O ; NCC(=O)O ; NCCC[C@H](N)C(=O)O ; "+" ; N=C(N)NCC(=O)O]

Guthmiller et al. (J Biol Chem. 1994 Jul. 1; 269 (26): 17556-60) have characterized a rat kidney AGAT by cloning and heterologously expressing the enzyme in Escherichia coli (E. coli). Muenchhoff et al. (FEBS Journal 277 (2010) 3844-3860) report the first characterization of an AGAT from a prokaryote also by cloning and heterologously expressing the enzyme in E. coli.

A microorganism capable of producing guanidinoacetic acid (GAA) was published by Zhang et al. (ACS Synth. Biol. 2020, 9, 2066-275). They designed a reconstituted the ornithine cycle in E. coli by introducing a heterologous AGAT from different species (e.g., Homo sapiens, Cylindrospermopsis raciborskii, Moorea producens) and by introducing a citrulline synthesis module (e.g. ovexpression of carAB, argF and argI) and an arginine synthesis module (e.g. overexpression of argG, argH; introduction of aspA) into E. coli.

Schneider and Jankowitsch (WO2021122400 A1) propose a method to produce GAA using a microorganism having gene coding for a protein having the function of an L-arginine:glycine amidinotransferase and an increased carbamoyl phosphate synthase. The carbamoyl phosphate is an important precursor for the biosynthesis of GAA but also for L-arginine and other compounds. Furthermore, Wang et al. (Applied Microbiology and Biotechnology, 2021, vol. 105, pp. 3265-3276; https://doi.org/10.1007/s00253-021-11242-w) underlined among other that carbamoyl phosphate is essential for L-arginine production also in Corynebacterium sp.

Jankowitsch et al. (WO2022243116 A1) also disclose among other a Corynebacterium glutamicum strain for the production of guanidino acetic acid (GAA) expressing L-arginine:glycine amidinotransferase (AGAT; EC: 2.1.4.1) from Moorea producens, having deleted lysE, lysG and argR genes and overexpressing the carAB operon (encoding the carbamoyl-phosphate synthase). This microorganism may also have a guanidinoacetate N-methyltransferase activity in order to further produce creatine.

To increase the production of GAA using a microorganism an intracellular high amount of the starting materials arginine and/or glycine are necessary.

Glycine can be produced by organisms in the cell from various sources including L-serine. Glycine can be produced directly from L-serine by a L-serine hydroxymethyltransferase (SHMT) [EC 2.1.2.1] which is encoded in Escherichia coli (E. coli) and in Corynebacterium glutamicum (C. glutamicum) by the glyA gene. FIG. 1 shows a schematic depiction of glycine production from serine.

There are multiple publications focused on the production of L serine in various microorganisms. WO2016120326 A1 for example discloses a method for producing L-serine by cultivating a bacterium wherein the expression of the genes coding for polypeptides having serine deaminase activity and of a gene coding for a polypeptide having serine hydroxymethyltransferase activity, i.e. sdaA, sdaB, tdcG and glyA, respectively has been attenuated. CN109797126 A for example proposes for L-serine production the use of a bacterium having a reduced serine O-acetyltransferase (CysE) expression. CN113621638 A proposes a method for producing L-serine by fermentation of a bacterium having knocked-out genes related to an L-serine degradation pathway, such as serine hydroxymethyltransferase glyA, homoserine dehydrogenase thrA, serine dehydratase sdaA, serine dehydratase isozyme sdaB, L-serine transporter sdaC, serine dehydratase isozyme tdcG and threonine dehydrogenase tdcB genes.

WO2011080301 A2 discloses E. coli strains for the production of L-methionine. One of these strains comprises a missense mutation in the sdaA gene resulting in an increase of serine availability for the production of L-methionine. Another one of these strains comprises an additional copy of the glyA gene. The comparison to the strains having a mutated glyA gene leads to the conclusion that the glyA gene product catalyzes both, the conversion of L-serine to glycine and the degradation of L-threonine into glycine.

The object of the present invention is providing a microorganism having the capacity of producing GAA by using L-serine as a source of glycine and a method of producing GAA by cultivating said microorganism as well as a method for producing creatine.

Therefore, the present invention concerns a microorganism comprising at least one heterologous gene coding for a protein having the function of a L-arginine:glycine amidinotransferase and wherein a gene encoding a protein having the function of a L serine ammonia lyase [EC 4.3.1.17] (SDHL)), also referred to as L-serine deaminase or L-serine dehydratase activity, is inactivated or deleted.

A heterologous gene means that the gene has been inserted into a host organism which does not naturally have this gene. Insertion of the heterologous gene in the host is performed by recombinant DNA technology. Microorganisms that have undergone recombinant DNA technology are called transgenic, genetically modified or recombinant. A heterologous protein means a protein that is not naturally occurring in the microorganism. A homologous or endogenous gene means that the gene including its function as such or the nucleotide sequence of the gene is naturally occurring in the microorganism or is “native” in the microorganism. A homologous or a native protein means a protein that is naturally occurring in the microorganism.

Proteins having the function of an L-arginine:glycine amidinotransferase (AGAT) belong to the amidinotransferase family. The amidinotransferase family comprises glycine (EC: 2.1.4.1) and inosamine (EC: 2.1.4.2) amidinotransferases, enzymes involved in creatine and streptomycin biosynthesis respectively. This family also includes arginine deiminases, EC: 3.5.3.6. These enzymes catalyse the reaction:

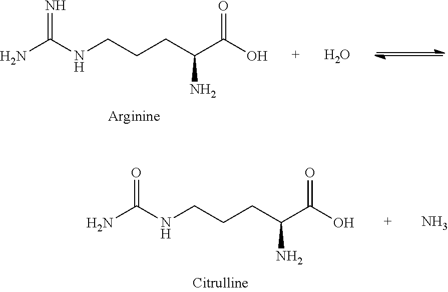

Also found in this family is the Streptococcus anti tumour glycoprotein. Enzymes or proteins with an L-arginine:glycine-amidinotransferase (AGAT) activity are also described to possess a conserved domain that belongs to the PFAM Family: Amidinotransferase (PF02274) (Marchler-Bauer A et al. (2017), “CDD/SPARCLE: functional classification of proteins via subfamily domain architectures.”, Nucleic Acids Res. 45 (D1): D200-D203.) as described also in the following publications: Pissowotzki K et al., Mol Gen Genet 1991; 231:113-123 (PUBMED: 1661369 EPMC: 1661369); D'Hooghe I et al., J Bacteriol 1997; 179:7403-7409 (PUBMED: 9393705 EPMC: 9393705); Kanaoka M et al., Jpn J Cancer Res 1987; 78:1409-1414 (PUBMED: 3123442 EPMC: 3123442).

In the microorganism of the present invention the gene coding for a protein having the function of an L-arginine:glycine amidinotransferase may further be overexpressed.

Overexpression of a gene is generally achieved by increasing the copy number of the gene and/or by functionally linking the gene with a strong promoter and/or by enhancing the ribosomal binding site and/or by codon usage optimization of the start codon or of the whole gene or a combination comprising a selection of all methods mentioned above.

In particular, overexpression of a gene can be achieved by increasing the copy number of the gene and/or by an enhancement of regulatory factors, e.g. by functionally linking the gene with a strong promoter and/or by enhancing the ribosomal binding site and/or by codon usage optimization of the start codon or of the whole gene. The enhancement of such regulatory factors which positively influence gene expression can, for example, be achieved by modifying the promoter sequence upstream of the structural gene in order to increase the effectiveness of the promoter or by completely replacing said promoter with a more effective or a so-called strong promoter. Promoters are located upstream of the gene. A promoter is a DNA sequence consisting of about 40 to 50 base pairs and which constitutes the binding site for an RNA polymerase holoenzyme and the transcriptional start point, whereby the strength of expression of the controlled polynucleotide or gene can be influenced. Generally, it is possible to achieve an overexpression or an increase in the expression of genes in bacteria by selecting strong promoters, for example by replacing the original promoter with strong, native (originally assigned to other genes) promoters or by modifying certain regions of a given, native promoter (for example its so-called -10 and -35 regions) towards a consensus sequence, e.g. as taught by M. Patek et al. (Microbial Biotechnology 6 (2013), 103-117) for Corynebacterium glutamicum (C. glutamicum). An example for a “strong” promoter is the superoxide dismutase (sod) promoter (“Psod”; Z. Wang et al., Eng. Life Sci. 2015, 15, 73-82). A “functional linkage” is understood to mean the sequential arrangement of a promoter with a gene, which leads to a transcription of the gene.

The genetic code is degenerated which means that a certain amino acid may be encoded by a number of different triplets. The term codon usage refers to the observation that a certain organism will typically not use every possible codon for a certain amino acid with the same frequency.

Instead, an organism will typically show certain preferences for specific codons meaning that these codons are found more frequently in the coding sequence of transcribed genes of an organism. If a certain gene foreign to its future host, i.e. from a different species, should be expressed in the future host organism the coding sequence of said gene should then be adjusted to the codon usage of said future host organism (i.e. codon usage optimization).

The microorganism of the present invention may further comprise an overexpressed gene encoding an enzyme having the function of a L-serine hydroxymethyltransferase (SHMT) [EC 2.1.2.1]. The L-serine hydroxymethyltransferase (SHMT) [EC 2.1.2.1] is encoded in Escherichia coli (E. coli) and in Corynebacterium glutamicum (C. glutamicum) by the glyA gene.

The GAA production starting from serine is shown in FIG. 2.

In the microorganism of the present invention the expression of an argR gene coding for the arginine responsive repressor protein ArgR may be inactivated or deleted.

In the context of the present invention, inactivation of a gene means that the gene is expressed at a low level or is not expressed, compared to that of a parent strain or an unmodified strain, or the activity is eliminated or decreased, even though the gene is expressed. In the present invention, the inactivation may be achieved by a mutation selected from insertion mutations in which one or more base pairs are inserted into the gene, or deletion mutations in which more than one base pair is deleted in the gene or by introducing one or more mutations in the group consisting of base pair transitions or transversion mutations of nonsense codons into the gene, or by replacing the native promoter of the gene by a weaker promoter of the gene.

The microorganism according to the present invention may further comprise at least one overexpressed gene encoding an enzyme having the function of a carbamoylphosphate synthase (EC 6.3.4.16, CarAB).

The microorganism of the present invention may further comprise at least one or more overexpressed genes selected from the group consisting of a gene coding for a protein having the function of an ornithine carbamoyltransferase (EC 2.1.3.3, ArgF, ArgF2), a gene coding for a protein having the function of an argininosuccinate synthetase (E.C. 6.3.4.5, ArgG), and a gene coding for a protein having the function of an argininosuccinate lyase (E.C. 4.3.2.1, ArgH).

In order to obtain relatively high L-arginine concentrations in the cell it is necessary to prevent L-arginine exportation. The amino acid exporter LysE counteracts the intracellular arginine concentration and reduces the substrate availability by efficiently transporting the substrate arginine from the cell. In addition, the citrulline from arginine biosynthesis is also secreted into the medium by an active LysE exporter. LysE is regulated by the transcriptional activator LysG (Bellmann, A., et al. (2001). “Expression control and specificity of the basic amino acid exporter LysE of Corynebacterium glutamicum.” Microbiology (Reading) 147 (Pt 7): 1765-1774).

In a particular embodiment of the present invention a lysEG gene coding for protein having the function of an arginine exporter LysE and its transcriptional activator LysG may be inactivated or deleted in the microorganism of the present invention.

The microorganism of the present invention may belong to the genus Corynebacterium, preferably Corynebacterium glutamicum (C. glutamicum), or to the genus Enterobacteriaceae, preferably Escherichia coli (E. coli), or to the genus Pseudomonas, preferably Pseudomonas putida (P. putida).

In Corynebacterium glutamicum the gene coding for the protein having the function of an arginine exporter is lysE and the gene coding for the transcriptional activator is lysG. In Escherichia coli the gene coding for the protein having the function of an arginine exporter is argO (ybjE). In Pseudomonas putida the protein having the function of an arginine exporter is lysE.

The protein having the function of an L-arginine:glycine amidinotransferase (AGAT) in the microorganism of the present invention may comprise an amino acid sequence which is at least 80% identical, preferably at least 90% identical to the amino acid sequence according to SEQ ID NO: 7. In a further embodiment of the present invention the amino acid sequence of the L-arginine:glycine amidinotransferase is identical to amino acid sequence according to SEQ ID NO: 7 of Moorea producens, a filamentous cyanobacterium.

The above-mentioned problem is further solved by a method for the fermentative production of guanidino acetic acid (GAA), comprising the steps of cultivating the microorganism according to the present invention as defined above in a suitable medium and accumulating GAA in the medium to form a GAA containing fermentation broth.

The method of the present invention may further comprise the step of isolating GAA from the fermentation broth.

The method according to the present invention may further comprise the step of drying and/or granulating the GAA containing fermentation broth.

The present invention further concerns a microorganism as defined above, further comprising a gene coding for an enzyme having the activity of a guanidinoacetate N-methyltransferase (EC: 2.1.1.2). Preferably, the gene coding for an enzyme having the activity of a guanidinoacetate N-methyltransferase is overexpressed.

The present invention also concerns a method for the fermentative production of creatine, comprising the steps of cultivating the microorganism according to the present invention comprising a gene coding for an enzyme having the activity of a guanidinoacetate N-methyltransferase in a suitable medium and accumulating creatine in the medium to form a creatine containing fermentation broth.

Preferably, the method further comprises isolating creatine from the creatine containing fermentation broth. creatine may be extracted from fermentation broth by isoelectric point method and/or ion exchange method. Alternatively, creatine can be further purified by a method of recrystallization in water.

### BRIEF DESCRIPTION OF THE FIGURES

FIG. 1: Schematic depiction of glycine production from serine via glycine by SHMT. Abbreviations: PGDH—Phosphoglycerate dehydrogenase [EC 1.1.1.95], PSAT—Phosphoserine transaminase [EC 2.6.1.52], PSP—phosphoserine phosphatase [EC 3.1.3.3], SDHL—L serine ammonia lyase [EC 4.3.1.17], SHMT—serine hydroxymethyltransferase [EC 2.1.2.1], 3P HP-3 phosphohydroxypyruvate, THF—(6S)-5,6,7,8-tetrahydrofolate, CH2-TFH—(6R)-5,10-methylene-5,6,7,8-tetrahydrofolate.

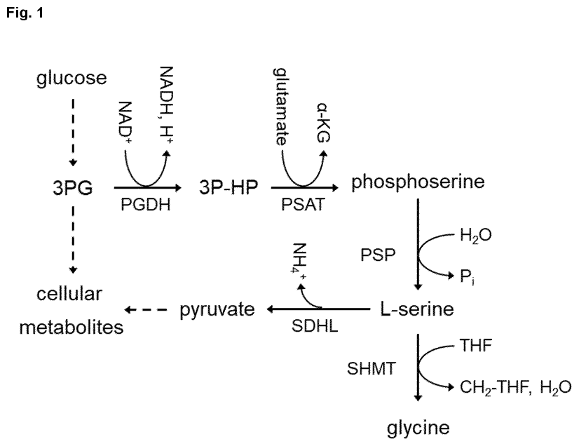

FIG. 2: Schematic depiction of GAA production from serine via glycine by SHMT and AGAT. Abbreviations: PGDH—Phosphoglycerate dehydrogenase [EC 1.1.1.95], PSAT—Phosphoserine transaminase [EC 2.6.1.52], PSP—phosphoserine phosphatase [EC 3.1.3.3], SDHL—L serine ammonia lyase [EC 4.3.1.17], SHMT—serine hydroxymethyltransferase [EC 2.1.2.1], AGAT—arginine:glycine amidinotransferase [E.C. 2.1.4.1], 3P HP-3 phosphohydroxypyruvate, THF—(6S)-5,6,7,8-tetrahydrofolate, CH2 TFH—(6R)-5,10-methylene-5,6,7,8-tetrahydrofolate.

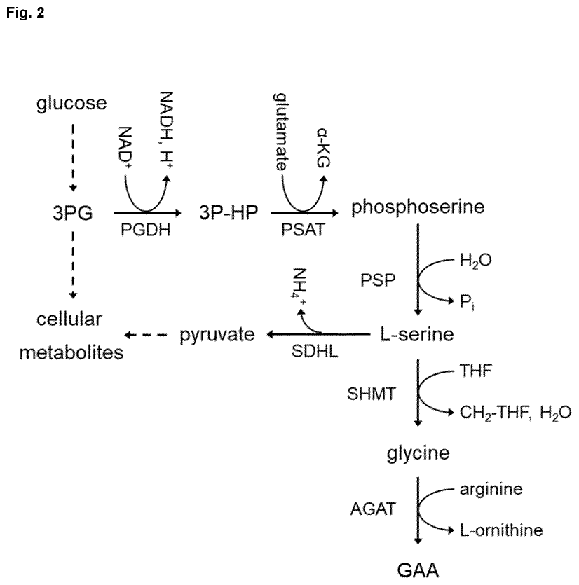

### BRIEF DESCRIPTION OF THE SEQUENCES

SEQ ID NO: 1 DNA sequence of the plasmid pK18mobsacB-sdaA
SEQ ID NO: 2 Synthetic oligonucleotide: DNA sequence of the primer oMC58
SEQ ID NO: 3 Synthetic oligonucleotide: DNA sequence of the primer oMC59
SEQ ID NO: 4 Synthetic oligonucleotide: DNA sequence of the primer oMC60
SEQ ID NO: 5 Synthetic oligonucleotide: DNA sequence of the primer oMC61
SEQ ID NO: 6 DNA sequence of the Moorea producens gene with locus_tag BJP34_00300. It encodes for an L-arginine:glycine amidinotransferase
SEQ ID NO: 7 Amino acid sequence of the L-arginine:glycine amidinotransferase of Moorea producens (NCBI Accession Number WP_070390602)
SEQ ID NO: 8 DNA sequence of the plasmid pLIB_P
SEQ ID NO: 9 DNA sequence of the plasmid pLIB_P [AGAT-Mp] (Example 1)

### EXPERIMENTAL SECTION

### A) Materials and Methods

### Chemicals

Unless explicitly stated, all chemicals were ordered from Merck, Sigma Aldrich, or Carl-Roth with analytical grade.

### Molecular Biology Techniques

All oligonucleotide primers were synthesized by Eurofins Genomics Germany GmbH (Ebersberg, Germany). Genomic DNA of C. glutamicum ATCC 13032 was isolated following manufacturer's instructions of the DNeasy Blood & Tissue Kits (Qiagen, Catalog #69504). DNA digestions were routinely performed using either High-Fidelity (HF®) restriction enzymes (NEB) or FastDigest Restriction Enzymes (FD; Thermo Fischer Scientific) following manufacturer's instructions.

Polymerase chain reaction (PCR) was used to amplify desired DNA regions with specific DNA oligos. Q5® High-Fidelity 2× Master Mix (NEB Catalog #M0492) and Phusion® High-Fidelity DNA Polymerase (NEB catalog #M0530), which have proof-reading activity, were used following the manufacturer's instructions. PCR and restriction fragments were cleaned up using the QIAquick PCR Purification Kit (Qiagen, Catalog #28106) according to the manufacturer's instructions. When necessary, purification of DNA fragments from an agarose gel was performed using the QIAquick Gel Extraction Kit (Qiagen, Catalog #28706), according to the manufacturer's instructions. Agarose gel electrophoresis was done using 0.8-1.2% agarose (Roth, Catalog #3810.4) dissolved in 1×TAE buffer (Roth, Catalog #CL83.3). Gels were cast with Roti® Gelstain (Roth, Catalog #3865.1) and electrophoresis itself was carried out at 150 V for 25-40 min as needed. DNA assembly of backbone and inserts for cloning of plasmids was done using the NEBuilder® HiFi DNA Assembly Master Mix (NEB, Catalog #E2621). Following DNA assembly, the reaction was transformed into E. coli competent cells to obtain individual clones (see DNA transformation). Correct assemblies were verified for the presence of the insert via a either colony PCR or restriction analysis and sequence integrity was confirmed by DNA sequencing. Taq DNA Polymerase (Qiagen, catalog #201203) or SapphireAmp® Fast PCR Master Mix (Takara, catalog #RR350) were used to confirm the presence of a desired DNA segment from transformed cells following manufacturer's instructions. Plasmid DNA after cloning were routinely isolated using one of the following kits following manufacturer's instructions: QIAprep Spin Miniprep Kit (Qiagen, Catalog #27106), HiSpeed Plasmid Kits (Qiagen, Catalog #12643). DNA sequencing was performed by either Eurofins Genomics Germany GmbH (Ebersberg, Germany) or Sequiserve GmbH (Vaterstetten, Germany).

### Strains and Cultivation Conditions

Corynebacterium glutamicum ATCC13032 (Kinoshita S, Udaka S, Shimono M., J. Gen. Appl. Microbiol. 1957; 3 (3): 193-205), the Corynebacterium glutamicum Type Strain/Wildtype, is commercially available at the American Type Culture Collection (ATCC) or at the DSMZ-German Collection of Microorganisms and Cell Cultures GmbH under the deposit no. DSM 20300.

Corynebacterium glutamicum ATCC 13032 and derivatives thereof were routinely grown at 30° C. in Brain heart infusion (BHI; Merck Millipore, Catalog #1104930500) broth or in CgXII minimal medium (Keilhauer et al., 1993) supplemented with 10 g/L glucose, 1 g/L L-arginine and 4 g/L L-serine, as specified. The exact composition of the CgXII minimal medium without any carbon sources is detailed in Table 1. Glycerol stocks of C. glutamicum made by mixing 900 μL of an overnight culture in BHI medium (30° C., 200 rpm, 10 mL medium) with 600 μL of sterile 86% (w/V) glycerol solution and then stored at −80° C. Escherichia coli strains were routinely grown at 37° C. in Lysogeny Broth (LB) medium (Sigma, Catalog #L3022-1 KG). Solid medium was supplemented with 1.5% (w/v) agar. Antibiotics were used for selection purposes when needed at the following concentrations: chloramphenicol—34 mg/L for E. coli and 7.5 mg/L for C. glutamicum; kanamycin—25-50 mg/L for E. coli and 15-25 mg/L for C. glutamicum.

TABLE 1
Composition of CgXII minimal medium without carbon sources

| Component | Final concentration in medium | |
|---|---|---|
| (NH₄)₂SO₄ | 20 | g/L |
| Urea | 5 | g/L |
| K₂HPO₄ | 1 | g/L |
| KH₂PO₄ | 1 | g/L |
| MOPS | 42 | g/L |
| MgSO₄•7H₂O | 0.25 | g/L |
| CaCl₂•2H₂O | 13.2 | mg/L |
| FeSO₄•7H₂O | 10 | mg/L |
| MnSO₄•H₂O | 10 | mg/L |
| ZnSO₄•7H₂O | 1 | mg/L |
| CuSO₄ | 0.2 | mg/L |
| NiCl₂•6H₂O | 0.02 | mg/L |
| Biotin | 0.2 | mg/L |
| Protocatechuic acid | 2.5 | g/L |

### DNA Transformation

E. coli chemical or electrocompetent cells were used for cloning and amplification of plasmid DNA (NEB, Catalog #C3019, C3020, C3040) following the manufacturer's instructions. After the recovery period, cells were spread on LB agar plates with the appropriate antibiotic for selection.

C. glutamicum strains were made electrocompetent using a modified protocol: a 100 mL culture of BHI+0.5 M Sorbitol was inoculated from an overnight preculture grown at 33° C., 250 rpm to an OD₆₀₀ of 0.3 and incubated at 33° C., 130 rpm until it reached an OD₆₀₀ of 1.75. The culture was transferred to 50 mL falcon tubes and collected by centrifugation (3400 g, 10 min, 4° C.). The cell pellet was combined and washed twice with 50 mL of ice-cold Tris-glycerol buffer (1 mM Tris-HCl pH 7.5, 10% (v/v) glycerol), followed by 2 wash steps with 50 mL of ice-cold 10% (v/v) glycerol. The final cell pellet was resuspended in 800 μL of ice-cold 10% (v/v) glycerol. Electrocompetent cells were aliquoted as 150 UL and stored at −80° C. For electroporation, an aliquot of cells was thawed on ice and DNA was added to it. The cells-DNA mixture was transferred to a 2 mm electroporation cuvette (Sigma-Aldrich, Catalog #Z706088-50EA) and a 2.5 kV, 200 Ω, 25 μF pulse with a 5 ms time constant was triggered using a Gene Pulser Xcell™ (Bio-Rad). The cuvette was placed on ice briefly and the contents were transferred to 4 mL of BHI (prewarmed to 46° C.) and a 6 min heat shock at 46° C. was applied to the cells. Cells were recovered at 30° C., 200 rpm for 50 min in the BHI medium. After recovery, the cells were spread on BHI agar plates with the appropriate antibiotic for selection and incubated at 30° C. for 2-3 days to get single cell colonies. Correct maintenance of replicative plasmids after transformation was verified by plasmid DNA isolation using the QIAprep Spin Miniprep Kit (Qiagen, Catalog #27106X4) following manufacturer's instructions with the exception that cells resuspended in Buffer P1 and incubated at 37° C. for 2 h before lysis. Isolated plasmid DNA was then verified by restriction analysis.

Allelic Exchange in C. glutamicum

Allele replacements, both for gene deletion and integration of genes at defined loci, were done using pK18mobsacB (NCBI Accession Number: FJ437239) (Schafer et al., 1994) derivative plasmids containing an upstream and downstream homologous region (~300-1000 bps) as well as the desired insert in the case of integration. The appropriate plasmid was transformed into C. glutamicum strains by electroporation as previously mentioned. After electroporation, the first recombination event, yielding an intermediate strain, was selected for on BHI agar medium containing kanamycin. Intermediate strains were screened via colony PCR. Next, intermediate strains were counter selected on BHI+10% (w/v) sucrose without antibiotic agar plates to promote the second recombination event. After counterselection, sucrose resistant and kanamycin sensitive colonies were screened by colony PCR and DNA sequencing using appropriate primers outside of the homologous regions to verify the desired allelic exchange took place.

Cultivation of C. glutamicum Strains for GAA Production for Example 3

The appropriate C. glutamicum strains were used to inoculate precultures from glycerol stocks. Precultures were done in 10 mL of BHI medium, with the appropriate antibiotic as needed, in 100 mL baffled Erlenmeyer flasks and incubated at 30° C., 200 rpm for 24 h. Precultures were collected by centrifugation (3100 g, 10 min, at room temperature (RT)) and washed with 5 mL of CgXII medium without carbon sources (see Table 1). The washed pellet was resuspended in 2.5 mL of CgXII medium without carbon sources and the OD₆₀₀ of the cell suspension was determined using an Ultraspec 2100 pro (Amersham Biosciences) spectrophotometer. The appropriate volume of washed cells was used to inoculate CgXII medium supplemented with 10 g/L glucose, 1 g/L L-arginine, 1.275 g/L L-ornithine and 3 g/l glycine with the appropriate antibiotic as needed, to a start OD₆₀₀ of 0.5 as the main culture. The main cultures were transferred to a 48-well multitier plate (Beckman Coulter Life Sciences, Catalog #M2P-MTP-48-BOH1) using 800 μL per well and the plate was covered with a sealing foil (Beckman Coulter Life Sciences/m2p Labs, Catalog #F-GPR48-10).

The covered plate was placed in a BioLector® | (Beckman Coulter Life Sciences/m2p Labs) for incubation at 1400 rpm, 30° C. for 27 h. After 27 h, the OD₆₀₀ was measured in a spectrophotometer, the cultures were spun down (2000 g, 10 min), and supernatants were used to measure GAA production.

### Quantification of GAA

GAA from culture supernatants were quantified by HPLC-UV on a Dionex Ultimate 3000 System (Thermo Scientific). Samples were filtered with Sartorius Minisart NML Plus 0.2 μm (Sartorius AG, Catalog #ST17823-K) and then diluted appropriately in Mobile phase A (see below). Next, samples (10 μL injection volume) were separated using a HyperCarb 100×4.6 mm 7 μm column (Thermo Scientific catalog no. 35007-104630) at 35° C. Mobile phase A consisted of 2.3 g Ammonium dihydrogen phosphate and 2.6 g di-Ammonium hydrogen phosphate dissolved in 2 L of purified water. Mobile phase B consisted of 2.3 g Ammonium dihydrogen phosphate and 2.6 g di-Ammonium hydrogen phosphate dissolved in 1.25 L of purified water and 0.75 L acetonitrile. A flow rate of 1.0 mL min-1 was maintained constant throughout the run. The column was equilibrated with 100% mobile phase A beforehand and a gradient between phases A and B was used: 0 to 8 min—phase B linear gradient increased from 0-10%; 8 to 10 min—linear gradient increased from 10-40% B; 10 to 11 min—40% B; 11.1 to 13 min—0% B, and 13 to 14 min—constant at 0% B to re-equilibrate the column. The total run duration was 14 min. Under these conditions, GAA had a retention time of 5.8 min. GAA was detected at 200 nm (210 nm as reference) using a UV Detector (ThermoScientific Dionex Ultimate 3000 DAD).

### B) Experimental Results

### Example 1: Construction of sdaA Inactivation Strain in C. glutamicum

To improve L-serine availability by preventing its conversion to pyruvate, the sdaA gene of C. glutamicum was inactivated. The sdaA gene of C. glutamicum encodes for a protein with L-serine ammonia-lyase (EC 4.3.1.17) activity (abbreviated as SDHL). Alternative names of SDHL are serine deaminase, L-serine dehydratase, and L-serine deaminase, amongst others. Inactivation was achieved via allele replacement with the plasmid pK18mobsacB-sdaA (SEQ ID NO: 1) in C. glutamicum ATCC 13032 yielding the strain C. glutamicum ATCC 13032 ΔsdaA.

pK18mobsacB-sdaA was cloned as follows:
Backbone: BamHI, Sall linearized pK18mobsacB.
Insert(s): PCR of ATCC 13032 gDNA with oligos oMC58 (SEQ ID NO: 2) and oMC59 (SEQ ID NO: 3), PCR of ATCC 13032 gDNA with oligos oMC60 (SEQ ID NO: 4) and oMC61 (SEQ ID NO: 5). Assembled via HiFi Assembly mix.

For inactivating sdaA, pK18mobsacB-sdaA was transformed into C. glutamicum ATCC 13032 by electroporation. Chromosomal integration (resulting from a first recombination event) was selected by plating on BHI agar supplemented with 134 g/l sorbitol, 2.5 g/l yeast extract and 25 mg/l kanamycin. The agar plates 10 were incubated for 48 h at 33° C. Individual colonies were transferred onto fresh agar plates (with 25 mg/l kanamycin) and incubated for 24 h at 33° C. Liquid cultures of these clones were cultivated for 24 h at 33° C. in 10 ml BHI medium contained in 100 ml Erlenmeyer flasks with 3 baffles. To isolate clones that have 15 encountered a second recombination event, an aliquot was taken from each liquid culture, suitably diluted and plated (typically 100 to 200 μl) on BHI agar supplemented with 10% saccharose. These agar plates were incubated for 48 h at 33° C. Colonies growing on the saccharose containing agar plates were then examined for kanamycin sensitivity. To do so a toothpick was used to remove cell material from the colony and to transfer it onto BHI agar containing 25 mg/l kanamycin 20 and onto BHI agar containing 10% saccharose. The agar plates were incubated for 60 h at 33° C. Clones that proved to be sensitive to kanamycin and resistant to saccharose were examined by PCR and DNA sequencing. The resulting strain was named C. glutamicum ATCC 13032 ΔsdaA.

### Example 2: Plasmid-Based Expression of AGAT

A heterologous gene encoding for an L-arginine:glycine amidinotransferase (AGAT), EC 2.1.4.1, is required for the formation of guanidinoacetic acid (GAA) from of L-arginine and glycine. Moorea producens is a filamentous cyanobacterium. The genome of the M. producens strain PAL-8-15-08-1 can be accessed under Genbank accession Number CP017599.1 (Leao et al., 2017). It contains an open reading frame coding for a L-arginine:glycine amidinotransferase (AGAT, EC 2.1.4.1; locus_tag BJP34_00300 shown in SEQ ID NO: 6). SEQ ID NO: 7 shows the corresponding amino acid sequence (NCBI Accession Number WP_070390602). The AGAT-Mp expression cassette was assembled into a plasmid as an intermediate step to facilitate future cloning yielding pLIB_P [AGAT-Mp] (SEQ ID NO: 9). pLIB_P [AGAT-Mp] contains a promoter sequence, ribosomal binding site, and a codon-optimized version of the AGAT-Mp gene. The corresponding gene product has the same amino acid sequence given in SEQ ID NO: 7.

Plasmid pLIB_P (SEQ ID NO: 8), comprises the replication origin from pBL1 for C. glutamicum, the pSC101 replication origin for E. coli, a kanamycin resistance gene, a strong promoter (P_{294MU} from (Rytter et al., 2014)), and the BioBricks Terminator BBa_B1006.

The codon-optimized version of the AGAT-Mp gene including an RBS and flanked by Eco31I (Bsal) sites was ordered from Eurofins Genomics. It was delivered as a synthetic gene cloned in pEX-A258. The codon-optimized AGAT-Mp was cloned into pLIB_P yielding pLIB_P [AGAT-Mp].

pLIB_P [AGAT-Mp] was cloned as follows:
Backbone: Eco31I linearized pLIB_P.
Insert: 1.3 kb fragment of Eco31I digest of pEX-A258-AGAT-Mp.
Assembled via HiFi Assembly mix.

The pLIB_P [AGAT-Mp] plasmid was transformed into strains C. glutamicum ATCC 13032 ΔsdaA (created in Example 1) and strain C. glutamicum ATCC 13032 (reference strain) to test GAA production from these strains. Plasmid containing cells were selected with 25 mg/l kanamycin. Confirmed strains were named C. glutamicum ATCC 13032 pLIB_P [AGAT-Mp] and C. glutamicum ATCC 13032 ΔsdaA pLIB_P [AGAT-Mp], respectively.

### Example 3: Impact of the Inactivation of L-Serine Ammonia-Lyase (SDHL) and Implementation of L-Arginine: Glycine Amidinotransferase (AGAT) on GAA Production

To assess the impact of the inactivation of L-serine ammonia-lyase (SDHL) and implementation of L-arginine:glycine amidinotransferase (AGAT) on GAA production, strains C. glutamicum ATCC 13032 pLIB_P [AGAT-Mp] and C. glutamicum ATCC 13032 ΔsdaA pLIB_P [AGAT-Mp], were cultivated in the BioLector system in CGXII medium, and the resulting GAA titers were determined.

TABLE 2
GAA production of C. glutamicum ATCC 13032 ΔsdaA
with L-arginine:glycine amidinotransferase (AGAT).

| | GAA |
|---|---|
| | produced |
| Strain | (mg/L) |
| C. glutamicum ATCC 13032 pLIB_P[AGAT-Mp] | 193 |
| C. glutamicum ATCC 13032 ΔsdaA pLIB_P[AGAT-Mp] | 220 |

Table 2 shows that C. glutamicum ATCC 13032 ΔsdaA pLIB_P [AGAT-Mp] produces 220 mg/L of GAA which is an improved GAA production compared to 193 mg/L of GAA using the reference strain, C. glutamicum ATCC 13032 pLIB_P [AGAT-Mp].

Thus, we conclude that the inactivation of L-serine ammonia-lyase improves GAA production in a strain implementing L-arginine:glycine amidinotransferase (AGAT).

### CITATIONS

Jakoby, M., Ngouoto-Nkili, C.-E., & Burkovski, A. (1999). Construction and application of new Corynebacterium glutamicum vectors. Biotechnology Techniques, 13 (6), 437-441. https://doi.org/10.1023/A: 1008968419217
Keilhauer, C., Eggeling, L., & Sahm, H. (1993). Isoleucine synthesis in Corynebacterium glutamicum: molecular analysis of the ilvB-ilvN-ilvC operon. J Bacteriol, 175 (17), 5595-5603. https://doi.org/10.1128/jb.175.17.5595-5603.1993
Leao, T., Castelao, G., Korobeynikov, A., Monroe, E. A., Podell, S., Glukhov, E., Allen, E. E., Gerwick, W. H., & Gerwick, L. (2017). Comparative genomics uncovers the prolific and distinctive metabolic potential of the cyanobacterial genus Moorea. Proc Natl Acad Sci USA, 114 (12), 3198-3203. https://doi.org/10.1073/pnas.1618556114
Rytter, J. V., Helmark, S., Chen, J., Lezyk, M. J., Solem, C., & Jensen, P. R. (2014). Synthetic promoter libraries for Corynebacterium glutamicum. Appl Microbiol Biotechnol, 98 (6), 2617-2623. https://doi.org/10.1007/s00253-013-5481-x
Schafer, A., Tauch, A., Jager, W., Kalinowski, J., Thierbach, G., & Puhler, A. (1994). Small mobilizable multi-purpose cloning vectors derived from the Escherichia coli plasmids pK18 and pK19: selection of defined deletions in the chromosome of Corynebacterium glutamicum. Gene, 145 (1), 69-73. https://doi.org/10.1016/0378-1119 (94) 90324-7
Simic, P., Willuhn, J., Sahm, H., & Eggeling, L. (2002). Identification of glyA (encoding serine hydroxymethyltransferase) and its use together with the exporter ThrE to increase L-threonine accumulation by Corynebacterium glutamicum. Appl Environ Microbiol, 68 (7), 3321-3327. https://doi.org/10.1128/AEM.68.7.3321-3327.2002
Vrljic, M., Sahm, H., & Eggeling, L. (1996). A new type of transporter with a new type of cellular function: L-lysine export from Corynebacterium glutamicum. Mol Microbiol, 22 (5), 815-826. https://doi.org/10.1046/j.1365-2958.1996.01527.x

1. A microorganism, comprising:
at least one heterologous gene coding for a protein having the function of a L-arginine:glycine amidinotransferase, and
wherein
a gene encoding a protein having the function of a L-serine ammonia lyase is inactivated or deleted.

2. The microorganism of claim 1, further comprising:
an overexpressed gene encoding an enzyme having the function of a L-serine hydroxymethyltransferase.

3. The microorganism of claim 1, wherein an argR gene coding for the arginine responsive repressor protein ArgR is inactivated or deleted.

4. The microorganism of claim 1, further comprising:
at least one overexpressed gene encoding an enzyme having the function of a carbamoylphosphate synthase.

5. The microorganism of claim 1, further comprising:
at least one or more overexpressed genes selected from the group consisting of a gene coding for a protein having the function of an ornithine carbamoyltransferase, a gene coding for a protein having the function of an argininosuccinate synthetase, and a gene coding for a protein having the function of an argininosuccinate lyase.

6. The microorganism of claim 1, wherein a lysEG gene coding for a protein having the function of an arginine exporter LysE and its transcriptional activator LysG is inactivated or deleted.

7. A method for the fermentative production of guanidino acetic acid (GAA), the method comprising:
cultivating the microorganism as defined in claim 1 in a medium, and
accumulating GAA in the medium to form a GAA containing fermentation broth.

8. The method of claim 7, further comprising:
isolating GAA from the GAA containing fermentation broth.

9. A The microorganism as claimed in claim 1, further comprising:
a gene coding for an enzyme having the activity of a guanidinoacetate N-methyltransferase.

10. The microorganism of claim 9, wherein the gene coding for an enzyme having the activity of a guanidinoacetate N-methyltransferase is overexpressed.

11. A method for the fermentative production of creatine, the method comprising:
cultivating the microorganism as defined in claim 9, and
accumulating creatine in the medium to form a creatine containing fermentation broth.

12. The method of claim 11, further comprising:
isolating creatine from the creatine containing fermentation broth.
